(19) United States
(12) Patent Application Publication
(10) Pub. No.: US 2016/0288111 A1
(43) Pub. Date: Oct. 6, 2016

(54) Method For Synthesising Esters And Catalyst For Said Synthesis

(71) Applicants: PIVERT, Venette (FR); CENTER NATIONAL DE LA RECHERCHE SCIENTIFIQUE (CNRS), Paris Cedex 16 (FR); UNIVERSITÉ DES SCIENCES ET TECHNOLOGIES DE LILLE 1, Villeneuve d'Ascq Cedex (FR); ECOLE CENTRALE DE LILLE, Villeneuve D'ascq Cedex (FR)

(72) Inventors: Franck Dumeignil, Fretin (FR); Simon Desset, Lille (FR); Sébastien Paul, Thun Saint Amand (FR); Guillaume Raffa, Les Halles (FR); Lei Zhang, Villeneuve D'ascq (FR)

(21) Appl. No.: 15/035,444

(22) PCT Filed: Nov. 6, 2014
(86) PCT No.: PCT/FR2014/052839

§ 371 Date: May 9, 2016

(30) Foreign Application Priority Data
Nov. 8, 2013 (FR) 1360981

(51) Int. Cl. B01J 31/24; C07C 67/40; C07F 15/00; B01J 31/22

(52) U.S. Cl. CPC B01J 31/2414; B01J 31/2409; B01J 31/2282; C07C 67/40; C07F 15/0046; B01J 2231/49; B01J 2231/641; B01J 2531/821

(57) ABSTRACT

A method for synthesising esters from alcohols by dehydrogenating coupling in the presence of a catalyst of formula 1 as well as to the use of catalysts of formula 1 for synthesising esters. The method according to the invention can be used in particular for the production of dihydrogen. The invention also relates to novel catalysts as well as to the uses thereof.

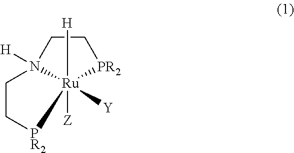

### FIELD OF THE INVENTION

The invention relates to a method for synthesizing esters from starting products, preferably biobased starting products, by dehydrogenative coupling in the presence of a catalyst. The starting products may be biobased alcohols such as fatty alcohols and/or polyols (for example glycerol) derived from oleaginous materials, or else may be alcohols produced by fermentation of biomass (for example ethanol and butanol).

### BACKGROUND OF THE INVENTION

Many esters, and especially ethyl acetate, are synthesized on the industrial scale by using starting products of fossil origin (ethylene in the case of ethyl acetate) via multi-step methods. The global market for ethyl acetate was 2.5 million tonnes/year in 2008.

It is known to use a ruthenium-based catalyst to carry out the dehydrogenative coupling of ethanol using for example carbonylchlorohydrido[bis(2-diphenylphosphinoethyl)amino]ruthenium(II), of formula A, (CAS: 1295649-40-9), Trade Name: Ru-MACHO, or D (see below) (cf. M. Nielsen, H. Junge, A. Kammer and M. Beller, Angew. Chem. Int. Ed., 2012, 51, 5711-5713 and EP 2 599 544 A1) or trans-RuCl₂(PPh₃)[PyCH₂NH(CH₂)₂PPh₂], of formula B, (cf. D. Spasyuk and D. Gusev, Organometallics, 2012, 31, 5239-5242). These catalysts A and B however require the presence of tBuOK or EtONa in order to be active. The catalyst D requires a long reaction time and a high catalytic loading in order to obtain yields of at most 90% ester.

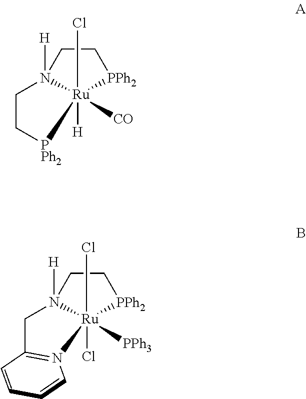

Another catalyst used for this same reaction is the carbonylhydrido(tetrahydroborato)[bis(2-diphenylphosphinoethyl)amino]ruthenium(II) catalyst of formula C (CAS: 1295649-41-0), Trade Name: Ru-MACHO-BH. This reaction is described in patent application WO 2012/144650 where this synthesis requires the presence of a hydrogen acceptor such as a ketone, for example 3-pentanone. In addition to dissolving the various species (catalyst and substrate), 3-pentanone acts as a hydrogen acceptor. Thus, an at least stoichiometric amount of 3-pentanone is used in the examples and the reactions described are therefore not accompanied by the release of gaseous hydrogen.

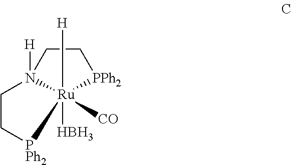

It is also known to use a ruthenium-based catalyst to carry out the dehydrogenative coupling of butanol using for example trans-RuH₂(CO)[HN(C₂H₄PiPr₂)₂], of formula D, (M. Bertoli, A. Choualeb, A. J. Lough, B. Moore, D. Spasyuk and D. Gusev, Organometallics, 2011, 30, 3479-3482) or [RuH(PNN)(CO)], of formula E, (cf. J. Zhang, G. Leitus, Y. Ben-David and D. Milstein, J. Am. Chem. Soc., 2005, 127, 10840-10841) in the presence of solvent and in the absence of base and hydrogen acceptor. However, these catalysts require long reaction times and high catalytic loadings in order to obtain yields of at most 90% ester.

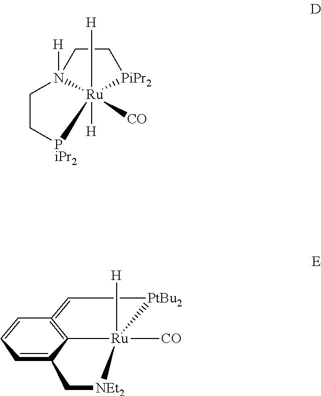

Setting up such methods on the industrial scale furthermore presents numerous disadvantages. One of these disadvantages is the need to carry out several purification steps in order to isolate the products of the reaction which makes the method significantly more complex. Another problem is the use of organic products, such as solvents, in addition to the starting products. The use of such products substantially increases the environmental impact of such syntheses, which should of course be avoided. A method has now been achieved that solves these problems and that thus offers a realistic alternative to the industrial methods for synthesizing esters using fossil resources. Indeed, this method makes it possible to combine high yields with simplified synthesis and purification steps in the implementation.

### SUMMARY OF THE INVENTION

The invention relates to a method for synthesizing an ester from an alcohol that comprises the use of a catalyst of formula 1 below, in the absence of ketones, aldehydes, alkenes, alkynes, sodium hydroxide, EtONa, MeONa or ^{t}BuOK.

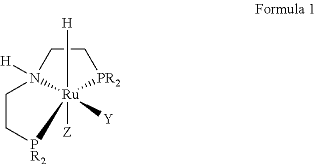

R, the R groups being identical or different, being an alkyl or aryl group, and more particularly a phenyl, isopropyl or cyclohexyl group; and
when R is an isopropyl group, Z is either a hydrogen atom, or a HBH₃ group, preferably Z is a HBH₃ group;
when R is a phenyl group, Z is either a hydrogen atom, or a HBH₃ group;
when R is a cyclohexyl group, Z is either a hydrogen atom, or a HBH₃ group;
Y being a CO carbonyl group. Alternatively, Y may be a PR′₃ phosphine, where R′ is a C₁-C₁₂ alkyl group or C₆-C₁₂ aryl group, more particularly a methyl, ethyl, i-propyl or phenyl group.

According to one preferred embodiment of the invention, said synthesis is carried out in the absence of a hydrogen acceptor compound and in the absence of base. This synthesis is therefore carried out without external addition of these compounds.

Thus, according to one particular embodiment, the invention relates to a method comprising the following step(s):
bringing an alcohol and a catalyst as defined in formula 1 into contact without addition of hydrogen acceptor compounds and without addition of bases.

The expression “hydrogen acceptor” denotes the organic compounds that are capable of reacting with molecular hydrogen, H₂, in order to form a novel compound under the reaction conditions for ester synthesis. In particular, it may be compounds such as ketones, aldehydes, alkenes and alkynes.

The absence of compounds that can react with the molecular hydrogen in order to absorb it is particularly advantageous since it enables in particular the production of gaseous hydrogen H₂ which is easily isolated from the reaction medium and which can thus be used subsequently. Furthermore, this avoids the stoichiometric formation of the hydrogenation product of the hydrogen acceptor, facilitating the downstream purification steps.

Thus, according to one preferred embodiment, the method according to the invention also makes it possible to obtain gaseous molecular hydrogen. This is separated from the reaction medium by simple phase separation and may be discharged and/or collected directly.

The term “base” denotes a compound capable of capturing one or more protons. Within the context of the invention, the term base very particularly denotes bases such as sodium hydroxide, or alkoxylated alkali metal salts in particular EtONa, MeONa or ^{t}BuOK.

Preferably, the reaction is carried out in the absence of toluene or xylene and in particular in the absence of solvent. The term solvent denotes a substance, liquid at its usage temperature, which has the property of dissolving, diluting or extracting alcohols and/or, optionally, the catalyst, without chemically modifying them under the reaction conditions for the synthesis of esters. Optionally, a solvent is not modified itself under the conditions of the reaction in which it participates. It may be compounds such as water, inorganic solvents, and organic solvents of hydrocarbon-based, oxygenated and halogenated type.

This solvent, in the absence of which the reaction is carried out, may obviously also be a hydrogen acceptor and/or a base. Thus, within the context of the invention, the term solvent especially denotes ketones, such as 3-pentanone, acetone or cyclohexanone. It also denotes aromatic or aliphatic hydrocarbon-based compounds which are optionally halogenated, ethers and alcohols. The expression “in the absence of” is used in its normal meaning which implies the absence, in the initial reaction mixture, of a sufficient amount of compound to play an effective role in the reaction and also the absence of external addition of this compound during the reaction. For example, the presence in the reaction medium of a minimum amount (for example in the form of a trace) of a hydrogen acceptor, of a base or of a solvent will not substantially influence the reaction. Thus, the absence of solvent implies the absence of an amount sufficient to dissolve/dilute/extract the starting product(s) (i.e. the alcohol(s)) and also the catalyst. For a solvent, this amount is generally greater than the numbers of moles of reactants, that is to say that the solvent is generally present in the reaction medium in a molar concentration of greater than or equal to 50%.

Preferably, the expression “absence of” implies a molar concentration of said compound of less than 10%, and more particularly of less than 5%, or even the complete absence thereof, i.e. less than 0.001%.

Preferably, the catalyst loading used in the method according to the invention is less than 10 000 ppm, more particularly less than 1000 ppm, even more particularly less than 500 ppm. This loading may for example be around 50±10 ppm.

Preferably, the catalyst loading used in the method according to the invention is selected from a range extending from 10 000 ppm to 1 ppm, more particularly from 1000 ppm to 10 ppm, even more particularly from 500 ppm to 50 ppm. This loading may for example be around 225±10 ppm.

The above proportions are given relative to the molar amount of starting products.

Preferably, the temperature of the reaction medium is selected from a temperature range extending from 200° C. to 15° C., more particularly from 150° C. to 40° C. and even more particularly from 130° C. to 80° C. This temperature may be around 130±1° C.

Preferably, the reaction is carried out at a pressure ranging from 20 bar to 1 bar. Advantageously, no particular pressure is applied and the reaction is carried out at atmospheric pressure and/or in an open system.

Preferably, the alcohol reacted with the catalyst of formula 1 is a primary alcohol.

Preferably, the alcohol reacted with the catalyst of formula 1 is a branched or non-branched, C1 to C30, in particular C2 to C6 or C8 to C22 and more particularly C16 to C18, primary alcohol. For example, it may be selected from the group consisting of ethanol, butanol, octan-1-ol, 2-ethyl-1-hexanol, nonan-1-ol, decan-1-ol, undecanol, lauryl alcohol, myristyl alcohol, cetyl alcohol, stearyl alcohol, docosanol, policosanol and mixtures thereof.

Preferably, the alcohol reacted with the catalyst of formula 1 is a linear fatty alcohol of formula:

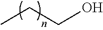

where n is ≧6, preferably the number of carbons of this fatty alcohol is an even number and n≧12;

or an alcohol derived from the fermentation of biomass where n is between 0 and 5 limits included, preferably where n=0 or 2, such as ethanol or butanol.

In the method according to the invention, the starting compound (alcohol) may be present alone or as a mixture with other reactants (i.e. other alcohols).

Furthermore, the starting compound may be used in purified form or in crude, especially unrefined, form, in particular when the compound is obtained from vegetable oils (e.g. from oleaginous materials). A crude alcohol is generally a composition, for example a distillate, that comprises no more than 90%, preferably no more than 80%, for example no more than 70% by volume of alcohol relative to the total volume of the composition. Thus, the starting product may be a composition comprising less than 95%, preferably less than 85%, for example less than 75% by volume of alcohol relative to the total volume of the composition.

According to one particular aspect of the invention, the method is carried out in the absence of any additive (other than the catalyst) that may have an effect on the coupling reaction of the alcohol and/or on the production of molecular hydrogen.

According to another preferred aspect of the invention, the method does not comprise a step of drying and/or degassing the alcohols. This is because it has surprisingly been determined that the catalyst (and particularly the catalysts where Z is HBH₃ such as Ru-MACHO-BH, of formula C)) remains active in the presence of air and traces of water.

Indeed it appears that, unexpectedly, these specific reaction conditions make it possible to simultaneously obtain a shorter reaction time and the use of a very reduced catalyst loading while retaining a high yield. The fact of being able to dispense with the use of additional chemical compounds such as those mentioned above makes it possible to reduce the manufacturing costs, to avoid the corrosion problems linked to their use and therefore to drastically reduce the environmental impact of such methods. This also makes it possible to greatly simplify the operations for separating and/or purifying the products of the reaction downstream of the method, operations such as the elimination of the solvent of the reaction by distillation, the neutralization of the reaction medium using a base, or the separation of the more numerous products of the reaction during the use of a hydrogen acceptor.

According to one particular aspect of the invention, the catalyst is preferably a catalyst of formula 1 in which the four R radicals are identical.

According to one particular aspect of the invention, the catalyst is preferably a catalyst of formula 1 in which Z is HBH₃ and/or Y is a CO radical and the R radicals are phenyl radicals.

According to one particularly preferred aspect of the invention, the catalyst used is a catalyst of formula 1 in which the Z group is H and the Y group is a PR′₃ phosphine where R′ is a C₁-C₁₂ alkyl group or C₆-C₁₂ aryl group, in particular a methyl, ethyl, isopropyl or phenyl group.

According to one preferred variant of the invention, the catalyst is the catalyst of formula C (R=Ph, Z═HBH₃, Y═CO).

According to one preferred variant of the invention, the catalyst is the catalyst of formula 1b (R=i-Pr, Z═HBH₃, Y═CO).

According to one preferred variant of the invention, the catalyst is the catalyst of formula 1c (R=Cy, Z═HBH₃, Y═CO).

According to one preferred variant of the invention, the catalyst is the catalyst of formula 6a (R=Ph, Z═H, Y═CO).

According to one preferred variant of the invention, the catalyst is the catalyst of formula 6c (R=Cy, Z═H, Y═CO).

The invention also relates to the catalysts described in this application as such and also to the methods for the manufacture thereof. In particular, a complex of formula 1c, 1b, 6a and 6c, and also a complex of the same formula where the carbonyl substituent is substituted by a phosphine, is one subject of the invention. The uses thereof in catalytic synthesis methods and such methods are also subjects of the invention. The catalytic synthesis methods may be hydrogenation reactions, alcohol amination reactions, amide synthesis reactions or Guerbet reactions.

The invention also relates to an ester obtained directly by the method described above.

### BRIEF DESCRIPTION OF THE DRAWINGS

The invention will be better understood on reading the appended figures, which are provided by way of examples and which are in no way limiting in nature, in which:

FIGS. 1a and 1b represent the change in the yield of ethyl acetate (FIG. 1a) and in the TON (FIG. 1b) as a function of time for the dehydrogenative coupling of ethanol for various loadings of catalyst C according to example 1a.

FIGS. 2a and 2b represent the change in the ester (butyl butyrate) yield of the synthesis according to the invention of example 1b (FIG. 2a) and in the Turn Over Number (TON=number of moles of substrate converted per mole of catalyst) (FIG. 2b) as a function of time.

FIGS. 3a and 3b represent the change in the ester (butyl butyrate) yield of the synthesis according to the invention of example 1c (FIG. 3a) and in the TON (FIG. 3b) as a function of time.

FIGS. 4a and 4b represent the change in the ester (butyl butyrate) yield of the synthesis according to the invention of example 1d (FIG. 4a) and in the TON (FIG. 4b) as a function of time.

FIGS. 5a and 5b represent the change in the ester (butyl butyrate) yield of the synthesis according to the invention of example 1e (FIG. 5a) and in the TON (FIG. 5b) as a function of time.

FIGS. 6a and 6b represent the change in the TON and yield of myristyl myristate as a function of time for the dehydrogenative coupling of tetradecanol according to the invention described in example 2.

### DETAILED DESCRIPTION OF THE INVENTION

### EXAMPLE 1

### Synthesis of Ethyl Acetate from Ethanol and of Butyl Butyrate from Butanol

The syntheses of ethyl acetate from ethanol and of butyl butyrate from butanol may be summarized by the following equations:

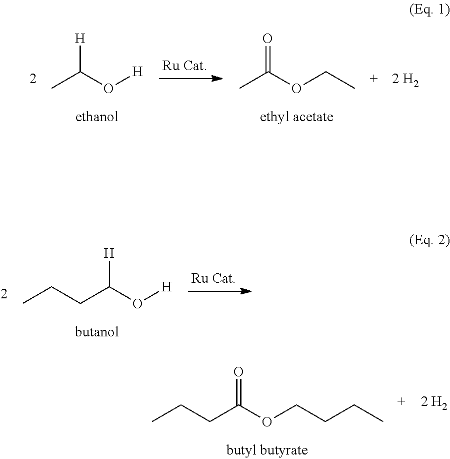

### EXAMPLE 1a

### Synthesis of Ethyl Acetate from Ethanol According to One Embodiment of the Invention Using a Catalyst of Formula C

50.2 mg (85.61 μmol) of catalyst of formula C ([Ru]≈500 ppm) is introduced into a Schlenk tube containing a stirrer bar. 7.8104 g (169.53 mmol) of ethanol is introduced via a syringe under an argon atmosphere. The Schlenk tube is then equipped with a condenser topped by a bubbler and an argon inlet. The system is heated to 78° C. with the aid of an oil bath and is stirred magnetically for 9 hours. Samples are withdrawn at various times with the aid of a syringe via a side inlet of the Schlenk tube. The samples are weighed, a known amount of internal standard (cyclohexane) is added and they are then diluted by dichloromethane.

The samples are analyzed by gas chromatography equipped with a flame ionization detector (GC-FID, Agilent Technologies 7890A, GC System, Zebron ZB-Bioethanol column) for determining the conversion and the selectivity of the reaction and also the material balance. The products are identified by gas chromatography equipped with a mass spectrometer (MS: Agilent Technologies 5975C VL MSD) and the results are compared with those of pure products. The results obtained are compiled in table I.

TABLE I
TOF (Turn Over Frequency = number of moles of
substrate converted per mole of catalyst per hour)
obtained for the dehydrogenative coupling of ethanol

| Catalyst | [Ru] (ppm) | TOF₀ (h⁻¹)^{a} | Yield (%)^{b} |
|---|---|---|---|
| C | 500 | 190 (9 h) | 75 (9 h) |

^{a}Calculated by linear regression of the variation of the TON as a function of time at the start of the reaction up to the time indicated between parentheses
^{b}Yield obtained after the time indicated between parentheses.

The coupling of ethanol to give ethyl acetate in the presence of the catalyst C takes place at a high rate, with ethyl acetate and traces of acetaldehyde (<1%) as product.

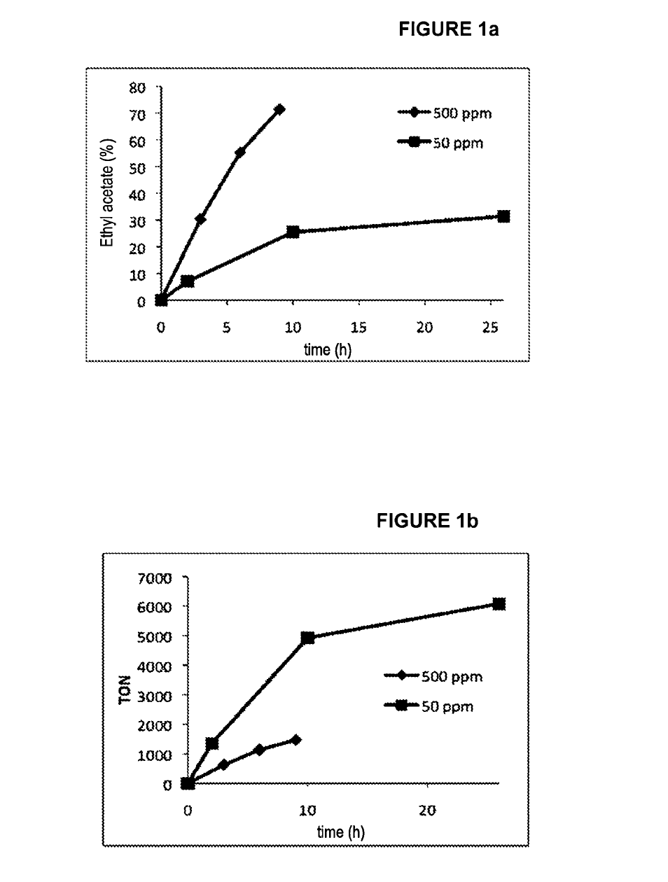

The coupling of ethanol to give ethyl acetate was studied for two different loadings of catalyst C, namely 50 and 500 ppm (table II, FIG. 1). The dehydrogenative coupling of ethanol may be carried out with a low catalyst loading, e.g. 50 ppm. Under these conditions, TOF₀ values of the order of 500 h⁻¹ are obtained and a TON of greater than 6000 after 26 h of reaction is subsequently observed.

TABLE II
Dehydrogenative coupling of ethanol catalyzed by various
loadings of catalyst C (T = 78° C.)

| [C] (ppm) | TOF₀ (h⁻¹) | TON | Ester (%) |
|---|---|---|---|
| 500 | 190 (9 h) | 1507 (9 h) | 75 (9 h) |
| 50 | 493 (10 h) | 6031 (26 h) | 31 (26 h) |

### EXAMPLE 1b

### Synthesis of Butyl Butyrate Using a Catalyst of Formula C according to One Embodiment of the Invention and Comparison with Methods from the Prior Art

16.1 mg (27.46 μmol) of catalyst of formula C ([Ru]≈240 ppm) is introduced into a Schlenk tube containing a stirrer bar. 8.5670 g (115.58 mmol) of butanol is introduced via a syringe under an argon atmosphere. The Schlenk tube is then equipped with a condenser topped by a bubbler and an argon inlet. The system is heated to 130° C. with the aid of an oil bath and is stirred magnetically for 5 hours. Samples are withdrawn at various times with the aid of a syringe via a side inlet of the Schlenk tube. The samples are weighed, a known amount of internal standard (cyclohexane) is added and they are then diluted by dichloromethane.

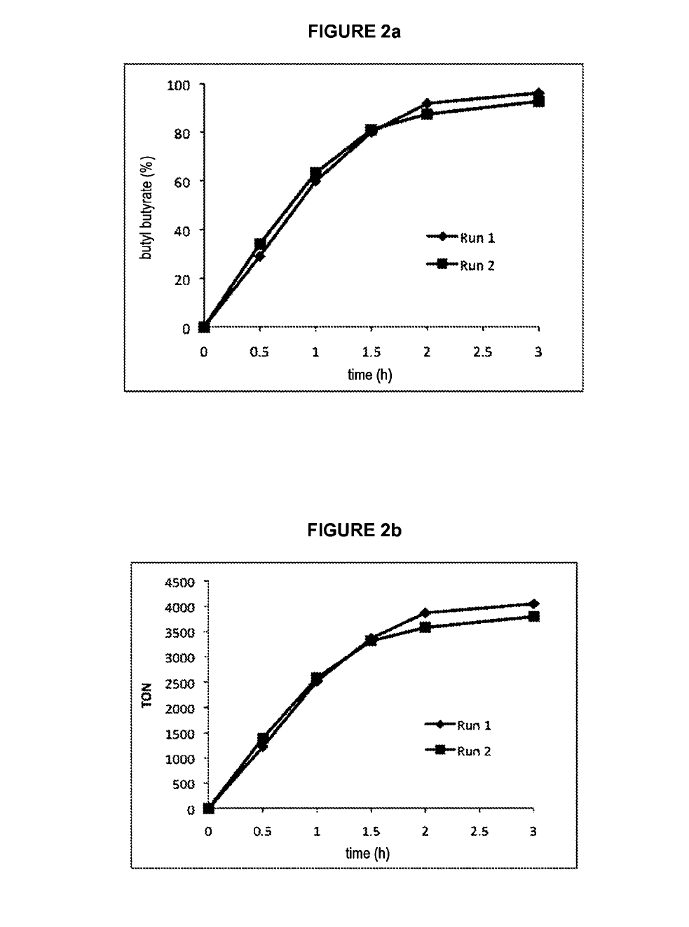

The samples are analyzed in the same way as for example la and the results obtained are compiled in table III and represented in FIGS. 2a and 2b.

The coupling of butanol to give butyl butyrate in the presence of the catalyst C takes place at a high rate, with butyl butyrate and traces of butyraldehyde (<1%) as product. The experiment was reproduced under the same conditions but this time with a catalyst: Ru-MACHO (or A). The results are compiled in table III:

TABLE III
Initial TOF values obtained for the catalyst C.

| Catalyst | TOF₀ (h⁻¹)^{a} |
|---|---|
| C | 2340 |
| A | 0 |

^{a}Calculated by linear regression of the variation of the TON as a function of time.

Surprisingly, the coupling of butanol to give butyl butyrate in the presence of the catalyst C in the absence of any additive and especially in the absence of base and solvent takes place at a high rate, with butyl butyrate and traces of butyraldehyde as product.

TABLE IV
Comparison between the results disclosed in example 31
of patent application WO 2012/144650 and the method described
above for the production of butyl butyrate.

| | BuOH/Ru in | | | | |
|---|---|---|---|---|---|
| Catalyst | mol/mol | T(° C.) | Solvent | Time | Ester (%) |
| C | 1000 | 120 | 3-pentanone | 9 h | 100 |
| C | 4150 | 118 | None | 3 h | 96 |

In addition to dissolving the various species (catalyst and substrate), 3-pentanone acts as a hydrogen acceptor. Thus, an at least stoichiometric amount of hydrogen acceptor (of solvent) is used in the examples from the literature and the reaction is therefore not accompanied by the release of two molecules of hydrogen per molecule of ester produced as in the method of the invention where the gaseous hydrogen is released from the reaction medium.

Besides this significant difference, the performances of the catalytic systems are markedly superior according to the method of the invention. These conditions make it possible to obtain, in times that are 3 times shorter and with a catalytic loading that is 4 times lower, a similar yield of butyl butyrate.

### EXAMPLE 1c

### Synthesis of Butyl Butyrate according to One Embodiment of the Invention and according to Equation 2 (Above) Using a Catalyst of Formula 6c

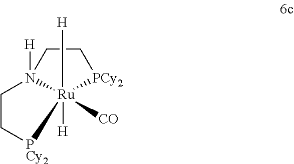

17 mg (28 μmol) of catalyst of formula 6c ([Ru]≈250 ppm) is introduced into a Schlenk tube containing a stirrer bar. 8.0450 g (108.54 mmol) of butanol is introduced via a syringe under an argon atmosphere. The Schlenk tube is then equipped with a condenser topped by a bubbler and an argon inlet. The system is heated to 130° C. with the aid of an oil bath and is stirred magnetically for 5 hours. Samples are withdrawn at various times with the aid of a syringe via a side inlet of the Schlenk tube. Samples are withdrawn at various times with the aid of a syringe via a side inlet of the Schlenk tube. The samples are then diluted by deuterated chloroform, CDCl₃.

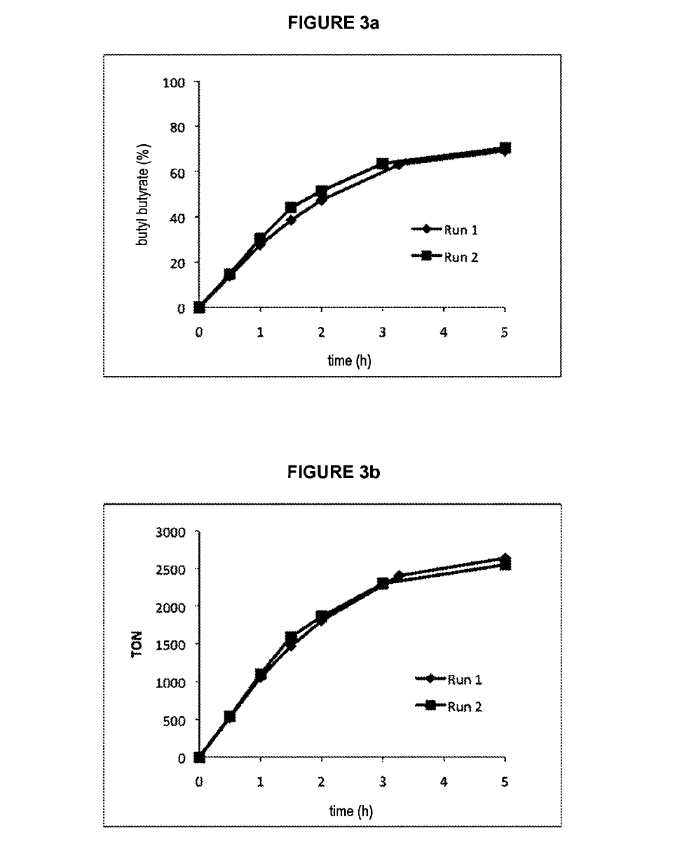

The yield is determined by a Bruker Avance 1, 300 MHz, 5 mm probe NMR spectrometer. The results obtained are compiled in table V and represented in FIGS. 3a and 3b.

TABLE V
Initial TOF values obtained

| Run | TOF₀/h^{-1 a} |
|---|---|
| 1 | 1005 |
| 2 | 1075 |

^{a} Calculated by linear regression of the variation of the TON as a function of time. In conclusion, the catalyst 6c is active for the dehydrogenative esterification of butanol in the absence of base and hydrogen acceptor.

### EXAMPLE 1d

### Synthesis of Butyl Butyrate according to One Embodiment of the Invention and according to Equation 2 (Above) Using a Catalyst of Formula 1b

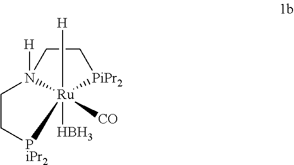

13 mg (25 μmol) of catalyst of formula 1 b ([Ru]≈260 ppm) is introduced into a Schlenk tube containing a stirrer bar. 8.1580 g (110.06 mmol) of butanol is introduced via a syringe under an argon atmosphere. The Schlenk tube is then equipped with a condenser topped by a bubbler and an argon inlet. The system is heated to 130° C. with the aid of an oil bath and is stirred magnetically for 5 hours. Samples are withdrawn at various times with the aid of a syringe via a side inlet of the Schlenk tube. The samples are weighed, a known amount of internal standard (cyclohexane) is added and they are then diluted by dichloromethane.

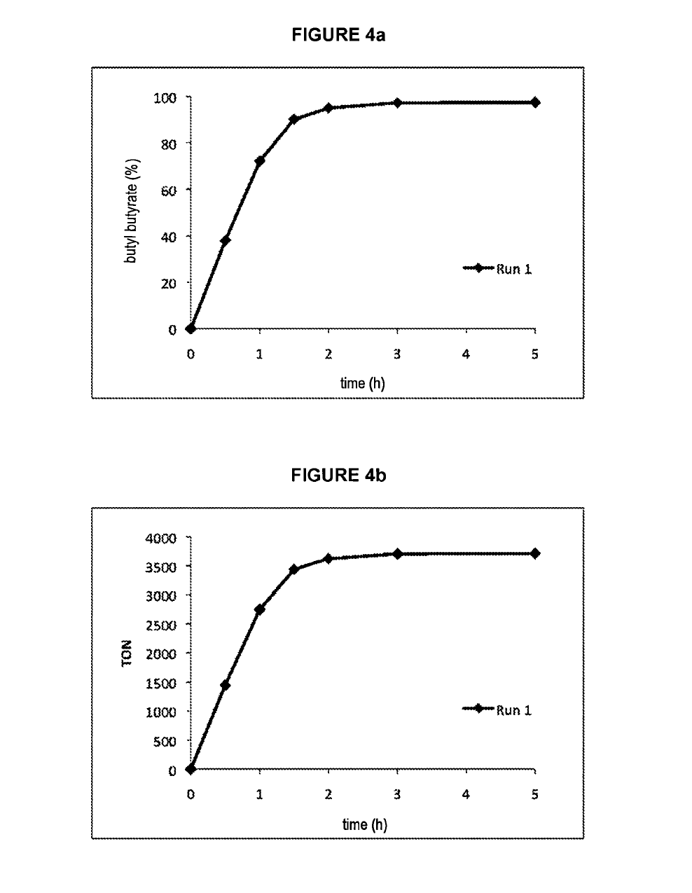

The samples are analyzed in the same way as for example la and the results are compared with those of pure products. The results obtained are compiled in table VI and represented in FIGS. 4a and 4b.

TABLE VI
Initial TOF values obtained

| Run | TOF₀/h⁻¹ ^{a} |
|---|---|
| 1 | 2465 |

^{a} Calculated by linear regression of the variation of the TON as a function of time.

In conclusion, the catalyst 1b is active for the dehydrogenative esterification of butanol in the absence of solvent, base and hydrogen acceptor.

### EXAMPLE 1e

### Synthesis of Butyl Butyrate according to One Embodiment of the Invention and according to Equation 2 (Above) Using a Catalyst of Formula 1c

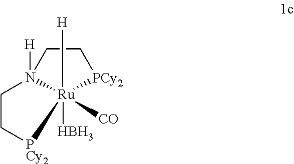

16 mg (26 μmol) of catalyst of formula 1c ([Ru]≈240 ppm) is introduced into a Schlenk tube containing a stirrer bar. 8.013 g (108.11 mmol) of butanol is introduced via a syringe under an argon atmosphere. The Schlenk tube is then equipped with a condenser topped by a bubbler and an argon inlet. The system is heated to 130° C. with the aid of an oil bath and is stirred magnetically for 5 hours. Samples are withdrawn at various times with the aid of a syringe via a side inlet of the Schlenk tube. Samples are withdrawn at various times with the aid of a syringe via a side inlet of the Schlenk tube. The samples are then diluted by deuterated chloroform, CDCl₃.

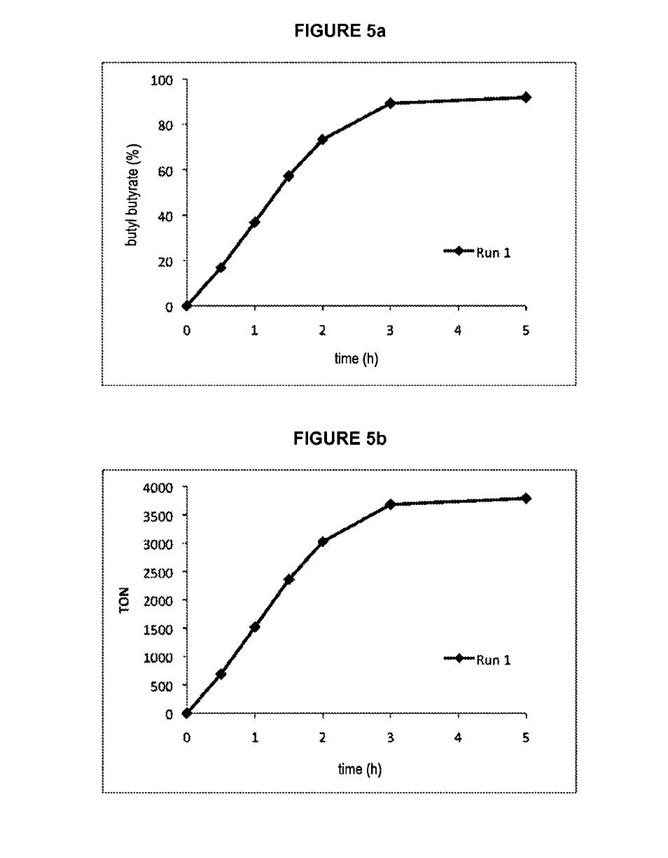

The yield is determined by a Bruker Avance 1, 300 MHz, 5 mm probe NMR spectrometer. The results obtained are compiled in table VII and represented in FIGS. 5a and 5b.

TABLE VII
Initial TOF values obtained

| Run | TOF₀/h^{-1 a} |
|---|---|
| 1 | 1545 |

^{a} Calculated by linear regression of the variation of the TON as a function of time. In conclusion, the catalyst 1c is active for the dehydrogenative esterification of butanol in the absence of solvent, base and hydrogen acceptor.

### EXAMPLE 2

### Synthesis of Myristyl Myristate from Tetradecanol

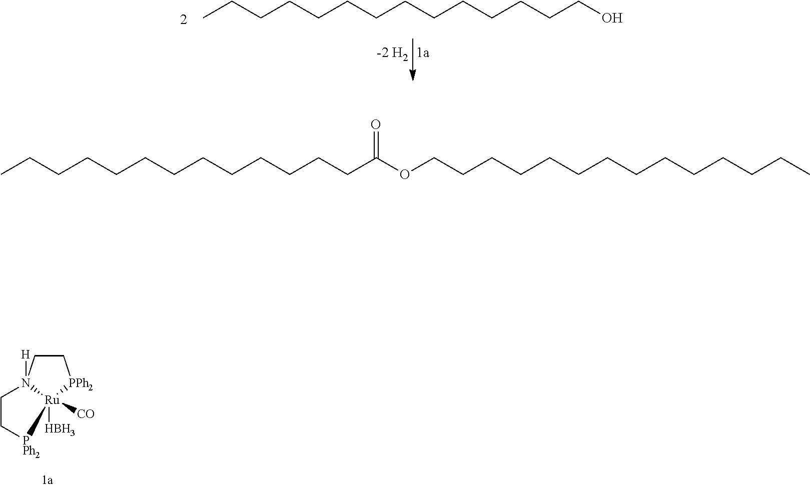

5.4 mg (9.21 μmol) of catalyst of formula C ([Ru]≈225 ppm) is introduced into a Schlenk tube containing a stirrer bar. 8.7662 g (40.89 mmol) of tetradecanol is introduced via a syringe under an argon atmosphere. The Schlenk tube is then equipped with a condenser topped by a bubbler and an argon inlet. The system is heated to 130° C. with the aid of an oil bath and is stirred magnetically for 6 hours. Samples are withdrawn at various times with the aid of a syringe via a side inlet of the Schlenk tube. The samples are then diluted by deuterated chloroform, CDCl₃.

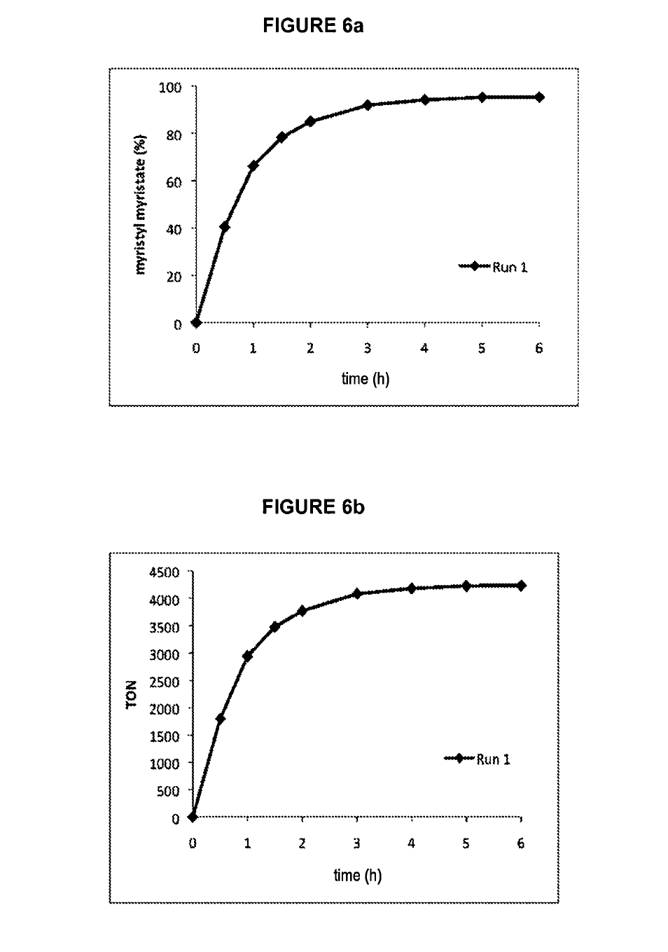

The yield is determined by a Bruker Avance 1, 300 MHz, 5 mm probe NMR spectrometer. The results are compiled in table VIII and represented in FIGS. 6a and 6b.

TABLE VIII
Initial TOF value obtained

| Run | TOF₀/h^{-1 a} |
|---|---|
| 1 | 3070 |

^{a} Calculated by linear regression of the variation of the TON as a function of time.

### EXAMPLE 3

### Synthesis of carbonylchlorohydrido[bis(2-diisopropyl-phosphinoethyl)amino]ruthenium(II) (2b) Starting Compound for the Synthesis of Compound 1b

The reaction scheme for the reaction is the following:

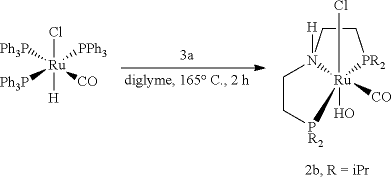

Synthesis: In a Schlenk tube, a suspension of carbonylchlorohydrido[tris(triphenylphosphine)]ruthenium(II) (Strem Chemicals, 0.999 g; 1.04 mmol) and of NH(C₂H₄PiPr₂)₂, 3a (0.357 g; 1.17 mmol) in diglyme (10 ml) is placed in an oil bath preheated to 165° C. and left stirring for two hours to give a clear yellow solution. The solution is left for 18 h at ambient temperature to give a precipitate. 10 ml of pentane are added and the suspension is cooled to 0° C. for 1 hour. The supernatant is then removed and the crystals are washed with diethyl ether (3×5 ml) and then dried under reduced pressure to give the desired product in the form of a very pale yellow powder. Yield: 64% (0.317 g).

³¹P-{¹H} NMR (CD₂Cl₂, 121.5 MHz): δ. 74.7 ppm. ¹H and ³¹P NMR in agreement with the spectral data from the literature. See: Bertoli, M.; Choualeb, A.; Lough, A. J.; Moore B.; Spasyuk, D.; Gusev D. G. Organometallics, 2011, 30, 3479.

### EXAMPLE 4

### Synthesis of carbonylchlorohydrido[bis(2-dicyclohexyl-phosphinoethyl)amino]ruthenium(II) (2c) Starting Compound for the Synthesis of Compounds 1c and 6c

The reaction scheme for the reaction is the following:

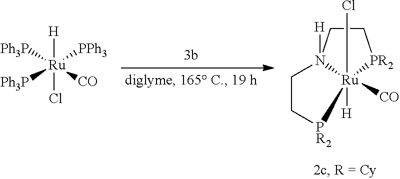

Synthesis: In a Schlenk tube, a suspension of carbonylchlorohydrido[tris(triphenylphosphine)]ruthenium(II) (Strem Chemicals, 1.000 g; 1.05 mmol) and NH(C₂H₄PCy₂)₂, 3b (0.498 g; 1.07 mmol) in diglyme (10 ml) is placed in an oil bath preheated to 165° C. and left stirring for 19 hours to give a suspension. The medium is then cooled to ambient temperature, the supernatant is removed and the precipitate is washed with diethyl ether (3×5 ml) and then dried under reduced pressure to give the desired product in the form of a very pale yellow powder. Yield: 78% (0.518 g). ³¹P-{¹H} NMR (CD₂Cl₂, 121.5 MHz): δ. 65.6 ppm. ¹H and ³¹P NMR in agreement with the spectral data from the literature. See: Nielsen, M.; Alberico, E.; Baumann, W.; Drexler H.-J.; Junge, H.; Gladiali, S.; Beller, M. Nature, 2013, in press (doi:10.1038/nature11891).

### EXAMPLE 5

### Synthesis of carbonylhydrido(tetrahydroborato)[bis(2-di-i-propylphosphinoethyl)amino]ruthenium(II)(1b)

The reaction scheme for the reaction is the following:

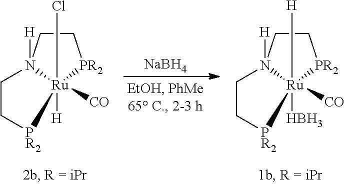

Synthesis: In a Schlenk tube under a stream of argon, a solution of NaBH₄ (5 mg; 0.24 mmol) in ethanol (2 ml) is added to a suspension of carbonylchlorohydrido[bis(2-di-i-propylphosphinoethyl)amino]ruthenium(II), 2b, (50 mg; 0.08 mmol) in toluene (8 ml). The Schlenk tube is then hermetically sealed, immersed in an oil bath preheated to 65° C. and left stirring for 2 h 30 min to give an opalescent solution. The solvent is then removed by distillation under reduced pressure (ambient temperature, 1×10⁻³ mbar). The white residue obtained is extracted using dichloromethane (3×5 ml) and filtered over a sintered glass. The filtrate is then concentrated under reduced pressure (ambient temperature, 1×10⁻³ mbar) to give the desired product in the form of a white powder (30 mg; yield: 62%).

¹H NMR (CD₂Cl₂, 300 MHz): δ. 3.90 (broad; 1H; NH); 3.30-3.12 (m, 2H); 2.56-2.44 (m, 2H); 2.30-2.21 (m, 2H); 1.94-1.82 (m, 2H); 1.38 (dd; J_{HP}=16.2 Hz; J_{HH}=7.5 Hz; 6H); 1.28-1.14 (m, 18 H); −1.92-−2.69 (broad; 4H; RuHBH₃); −13.53 (t, J_{HP}=17.7 Hz; 1H; RuH). ³¹P-{¹H} NMR (CD₂Cl₂, 121.5 MHz): δ. 77.7 ppm.

### EXAMPLE 6

### Synthesis of carbonylhydrido(tetrahydroborato)[bis(2-dicyclohexylphosphinoethyl)amino]ruthenium(II)(1c)

The reaction scheme for the reaction is the following:

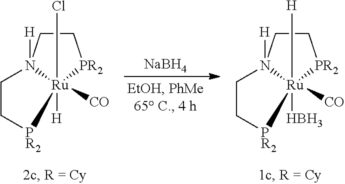

Synthesis: In a Schlenk tube under a stream of argon, a solution of NaBH₄ (48 mg; 1.37 mmol) in ethanol (12 ml) is added to a suspension of carbonylchlorohydrido[bis(2-dicyclohexylphosphinoethyl)amino]ruthenium(II), 2c, (200 mg; 0.43 mmol) in toluene (16 ml). The Schlenk tube is then hermetically sealed, immersed in an oil bath preheated to 65° C. and left stirring for four hours to give an opalescent solution. The solvent is then removed by distillation under reduced pressure (ambient temperature, 1×10⁻³ mbar). The white residue obtained is extracted using dichloromethane (3×5 ml) and filtered over a sintered glass. The filtrate is then concentrated under reduced pressure (ambient temperature, 1×10⁻³ mbar) to give the desired product in the form of a white powder (171 mg; yield: 88%).

¹H NMR (CD₂Cl₂, 300 MHz): δ. 3.85 (broad; 1H; NH); 3.27-3.09 (m, 2H); 2.27-2.10 (m, 8H); 1.96-1.68 (m, 20H); 1.61-1.20 (m, 22H); −2.19-−2.50 (broad; 4H; RuHBH₃); −13.60 (t, J_{HP}=17.9 Hz; 1H; RuH). ³¹P-{¹H} NMR (CD₂Cl₂, 121.5 MHz): δ. 68.9 ppm.

### EXAMPLE 7

### Synthesis of carbonyl(dihydrido)[bis(2-dicyclohexyl-phosphinoethyl)amino]ruthenium(II) (6c)

The reaction scheme for the reaction is the following:

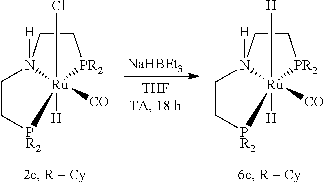

Synthesis: In a Schlenk tube, a whitish suspension of carbonylhydridochloro[bis(2-dicyclohexylphosphinoethyl)amino]ruthenium(II) 2c (63 mg; 0.1 mmol) in THF (2 ml) is treated with a 1 M commercial solution of NaHBEt₃ in toluene (0.12 ml; 0.12 mmol). Next, the medium is left stirring at ambient temperature under an argon atmosphere for 18 hours to give an opalescent light yellow solution. The solution is then concentrated under reduced pressure to result in a yellow solid residue which is dissolved with toluene (2 ml). This new solution is filtered over sintered glass and concentrated under reduced pressure to give a yellow powder. Yield: 80% (48 mg).

¹H NMR (C₆D₆, 300 MHz): δ. 2.32-2.07 (m, 6H); 1.90-1.60 (m, 31H), 1.34-1.03 (m, 16H); −6.18-−6.34 (m, 2H; RuH₂).

³¹P-{¹H} NMR (C₆D₆, 121.5 MHz): δ. 80.4 ppm.

The invention is not limited to the embodiments presented and other embodiments will become clearly apparent to a person skilled in the art.

1-5. (canceled)

6. A method for synthesizing an ester from an alcohol comprising a step of reacting at least one alcohol with a catalyst of formula 1: 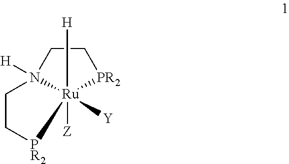
where R is a phenyl, isopropyl or cyclohexyl group, the R groups possibly being identical or different, and where:
when R is an isopropyl group, Z is a HBH₃ group;
when R is a phenyl group, Z is either a hydrogen atom, or a HBH₃ group; and
when R is a cyclohexyl group, Z is either a hydrogen atom, or a HBH₃ group;
and where Y is a CO carbonyl group or a PR′₃ phosphine group, R′ being a C₁-C₁₂ alkyl group or C₆-C₁₂ aryl group;
said synthesis being carried out in the absence of ketones, aldehydes, alkenes, alkynes, sodium hydroxide, EtONa, MeONa or ^{t}BuOK.

7. The method as claimed in claim 6, wherein said synthesis produces gaseous dihydrogen, H₂.

8. The method as claimed in claim 7, wherein said method comprises a step of capturing said gaseous dihydrogen.

9. The method as claimed in claim 6, wherein said at least one alcohol is a crude alcohol.

10. The method as claimed in claim 6, wherein said at least one alcohol is a branched or non-branched primary alcohol comprising a number of carbon atoms ranging from 1 and 30.

11. The method as claimed in claim 6, wherein that the reaction of said at least one alcohol and said catalyst is carried out in the absence of a hydrogen acceptor compound and in the absence of base.

12. The method as claimed in claim 6, wherein that said reaction is carried out in the absence of solvent.

13. The method as claimed in claim 6, wherein that the catalyst loading used is selected from a range extending from 10 000 ppm to 1 ppm.

14. The method as claimed in claim 6, wherein that the pressure of the reaction medium is atmospheric pressure or a lower pressure, and that the temperature of the reaction medium is selected from a range extending from 200° C. to 15° C.,

15. The method as claimed in claim 6, wherein that the alcohol reacted with the catalyst of formula 1 is an alcohol selected from the group consisting of ethanol, butanol, octan-1-ol, 2-ethyl-1-hexanol, nonan-1-ol, decan-1-ol, undecanol, lauryl alcohol, myristyl alcohol, cetyl alcohol, stearyl alcohol, docosanol, policosanol, and mixtures thereof.

16. The method as claimed in claim 6, wherein that the reaction may take place in the presence of air and traces of water.

17. A compound of formula 1b: 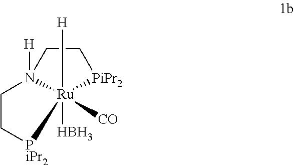
where iPr is an isopropyl group.

18. A compound of formula 1c: 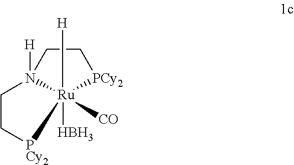
where Cy is a cyclohexyl group.

19. A compound of formula 6c: 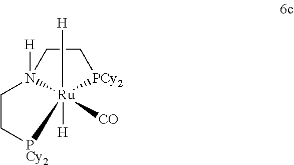
where Cy is a cyclohexyl group.

20. (canceled)

21. A method of catalytic synthesis comprising a hydrogenation step, said step being carried out by means of a catalyst of formula 1: 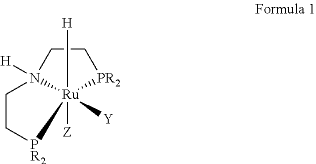
where R is a phenyl, isopropyl or cyclohexyl group, the R groups possibly being identical or different, and where:
when R is an isopropyl group, Z is a HBH₃ group;
when R is a phenyl group, Z is either a hydrogen atom, or a HBH₃ group; and
when R is a cyclohexyl group, Z is either a hydrogen atom, or a HBH₃ group;
and where Y is a CO carbonyl group or a PR′₃ phosphine group, R′ being a C₁-C₁₂ alkyl group or C₆-C₁₂ aryl group.
